(19) United States
(12) Patent Application Publication
(10) Pub. No.: US 2013/0317577 A1
(43) Pub. Date: Nov. 28, 2013

(54) THERAPEUTIC COOLING DEVICE

(71) Applicant: DOCTOR SLY LLC, (US)

(72) Inventor: Kyle E. Kingsley, Minneapolis, MN (US)

(73) Assignee: DOCTOR SLY LLC, Minneapolis, MN (US)

(21) Appl. No.: 13/874,861

(22) Filed: May 1, 2013

Related U.S. Application Data
(60) Provisional application No. 61/640,931, filed on May 1, 2012.

(51) Int. Cl. A61F 7/00

(52) U.S. Cl. CPC A61F 7/00
USPC 607/104; 607/109; 29/890.035

(57) ABSTRACT

A therapeutic cooling apparatus for cooling the human head that includes a container having a cavity that is configured to hold a volume of liquid and an indentation for supporting a human head. On one end of the container there is a contour and ramp that is designed to support the full length of the human neck when a person lies in the supine position and puts their head inside the cavity. Inside the container is a receptacle that is configured to hold at least one cooling insert. The receptacle is partitioned from the cavity by a divider. To use the apparatus, the cavity is filled with liquid. Cooling inserts are cooled and thereafter are placed within the receptacle. A person lowers their head and neck into the liquid-filled cavity so that their neck is supported by the contour and ramp and their head is supported by the indentation within the cavity. The head and neck are cooled by the passive transfer of heat from the head to the cooling insert through the liquid.

### FIELD

An apparatus and method to provide therapeutic cooling to a human head and neck are described.

### BACKGROUND

Cold therapy to the head and neck is used to alleviate pain associated with migraines, soothe sports injuries and concussions. In particular cold and darkness are used to treat migraine head pain. Common approaches to treat head pain include the application of ice packs or ice gels held directly to the head or neck by hand or by a securing apparatus. For example cooling packs and materials are applied directly to the head or are placed within head wraps, hats or the like. These techniques have limitations. These cooling apparatus often cool the head too quickly. Applying a cooling apparatus directly to the head or neck may also provide inadequate and/or asymmetrical cold therapy as areas of the head protrude into the cold pack or ice are disproportionally cooled as compared to recesses or less prominent areas of the head and the ice packs tend to have gaps or areas that are not given adequate therapy, even when held in place by a securing apparatus. Direct contact of cold packs or ice may also result in frost bite to areas of the skin directly exposed to the cold pack or ice. In view of the foregoing, it is clear that these traditional techniques are not perfect and leave room for more optimal approaches.

### SUMMARY

An improved cold therapy apparatus is described that cools the head and neck through indirect cooling, making it easier, safer and more thorough than the direct application of cold to the head and neck. Indirect cooling is defined herein as the passive diffusion of heat from the head and/or neck to a cooling source through an intermediate medium. The cold therapy apparatus comprises a container structure having at least one wall and a cavity defined therein that is configured to hold a volume of liquid. A contour exists on the wall that is configured to support at least a portion of a person's neck when that person lies on their back (the supine position) and places their head inside the cavity. The contour provides a seal about the person's neck to prevent liquid from exiting the cavity. An indentation on the bottom of the cavity is configured to support the back of the head. A ramp extends downwards from the contour to the indentation to provide further support along the neck. There is at least one receptacle within the container structure that is configured to hold at least one cooling insert. The receptacle may further comprise at least one stabilizing member that may be used to keep the cooling insert in place. A divider inside the cavity is configured to at least partially separate the receptacle from the cavity. The divider may also keep the head from sliding into the receptacle containing the cooling insert. The divider may also serve as a conduit for the transfer of heat between the liquid and the cooling insert.

In at least one embodiment, the contour of the apparatus can be extended outwards from the container to support the full length of the human neck and the upper shoulders. The contour may also contain protrusions to apply pressure to points on the neck, back and shoulders. The contour may provide further cooling therapy to the neck. In one embodiment, the contour may contain a small cut-away into which a cooling apparatus such as a gel pack can be inserted to follow the neck contour. The contour and/or indentation may contain padding to support the head or neck and/or a different texture to provide stimulation of the scalp and neck.

In at least one embodiment, an eye gel mask is detachably connected to the therapeutic apparatus for example by hook and loop material, snaps, protrusions, or any other suitable means of attachment.

In at least one embodiment, a wall extension extends upwards from the cavity to block additional light from the person's head. A blocking material could further be draped over the apparatus to further block light. The blocking material could be detachably connected to the apparatus via hook and loop material, snaps, protrusions or any other suitable means of attachment.

In at least one embodiment, the cooling insert comprises an ice pack or gel pack.

To use the therapeutic cooling apparatus, at least one cooling insert must be cooled. In one embodiment, the cooling insert is placed in the freezer which can be the cooling source. Once sufficiently cooled, at least one of the cooling inserts is placed into at least one receptacle within the apparatus. A person places the back of his neck and/or shoulders on the contour of the container and lowers the back of his head into the cavity so that the back of his head rests on the indentation at the bottom of the cavity. The ramp between the contour and indentation adds support along the length of the person's neck. The cavity is filled with liquid which can be the intermediate medium. At least a portion of the head inside the cavity is in contact with the liquid. In one embodiment the liquid inside the container reaches a person's ears when he is lying on his back.

Different amounts of liquid may be added to the cavity as long as the liquid does not exceed the lowest point of the contour (so that the liquid doesn't leak out). In one embodiment, the liquid level does not go above the ears. In another embodiment, approximately two quarts of liquid may be added to the cavity. A waterproof hairnet, e.g. a shower cap (not shown) may be used to prevent hair from getting wet. The liquid inside the cavity may vary in initial temperature. For example, the initial liquid temperature may be lukewarm, room temperature or cool. After the cooling insert(s) are added to the receptacle, the liquid inside the cavity cools from its initial temperature over the next several minutes or longer. Heat is conducted from the head to the liquid or other intermediate medium, into the cooling insert. In one embodiment, the intermediate medium inside the cavity is water. The head and/or neck are cooled by indirect cooling, e.g. the passive transfer of heat from the head to the cooling insert through the liquid.

In at least one embodiment, a person may cool an eye gel mask, and secure the cooled eye gel mask over the eyes so that it is used in tandem with the therapeutic cooling apparatus.

In at least one embodiment, a blocking material is placed over the sides of the therapeutic apparatus to block remaining light from reaching the head of the person.

In at least one embodiment, a cooling gel pack is inserted is added to the contour to provide direct cool therapy to the neck.

### BRIEF DESCRIPTION OF THE DRAWINGS

Reference is now made to the drawings in which like reference numbers represent corresponding parts throughout.

FIG. 1 is a perspective view of one embodiment of a therapeutic cooling apparatus.

FIG. 2 is a perspective view of the cooling apparatus in FIG. 1 with the addition of the cooling insert.

FIG. 3 is a perspective view of the cooling apparatus in FIG. 1 with the addition of a cut-out in the neck contour.

FIG. 4 is a perspective view of the cooling apparatus in FIG. 1 with the addition of an extension to the neck contour.

FIG. 5 is a front view of an eye gel mask.

FIG. 6 perspective view of the cooling apparatus in FIG. 1 with the addition of attachments for an eye gel mask.

FIG. 7 is a perspective view of the cooling apparatus in FIG. 1 in which the walls of the container are extended upwards from the cavity.

### DETAILED DESCRIPTION

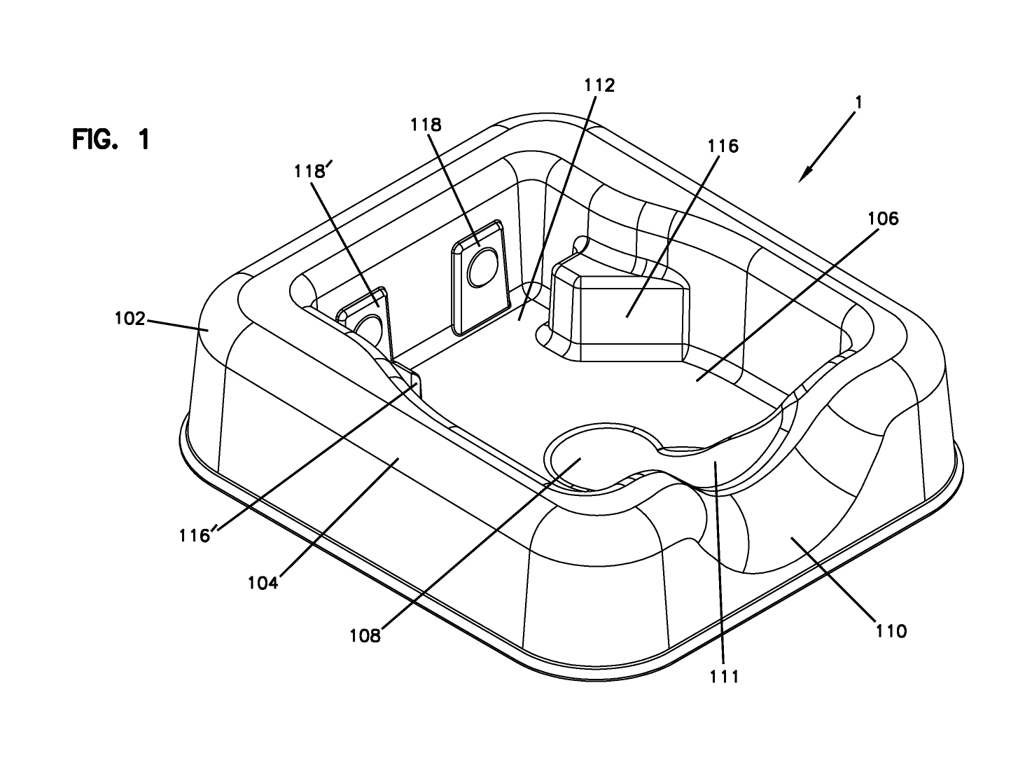

Pending U.S. application Ser. Nos. 13/208,324 and 13/208,334 are incorporated herein by reference. With reference to FIGS. 1-7, a therapeutic cooling apparatus is illustrated that uses indirect cooling to cool the neck and/or head through the passive diffusion of heat from the head and/or neck to a cooling source through an intermediate medium. A therapeutic cooling apparatus 100 comprises a container 102 having at least one wall 104 and a cavity 106 configured to hold a volume of liquid. A contour 110 is formed in the wall 104 of the container that is configured to support at least a portion of the human neck if a person were to place the back of his head inside the cavity 106 when in the supine position. The contour 110 can provide a seal about a portion of the person's neck to prevent liquid (not shown) from exiting the cavity 106 when the person's head displaces a volume of liquid. An indentation 108 is configured on the bottom of the cavity 106 to at least partially support a human head. A ramp 111 extends downward from the contour 110 to the indentation 108 to add further support along the length of the neck. The contour 110, ramp 111 and/or indentation 108 may be padded with additional material and/or air to provide additional comfort for the back of the head and/or neck. The cavity 106 is large enough to hold at least a portion of a human head and/or neck when the cavity 106 is filled with a liquid. In one embodiment, the cavity 106 is filled with liquid so that the liquid contacts the back of the head. In another embodiment, the liquid in the cavity 106 touches a person's ears, e.g. when the back of a person's head is resting in the cavity 106. In another embodiment, the liquid in the cavity 106 covers the top of the head. Any liquid can be used to fill the cavity 106 as long as it can be cooled, is safe for the anatomy of the human head and neck, and can serve as an intermediate medium to through which heat may transfers from the head to the cooling source. The container 102 can be made from any liquid impermeable material that is capable of at least partially supporting the weight of a human head and/or neck. Non limiting examples of liquid impermeable materials include plastic or other rigid semi/rigid material such as rubber, plastic or neoprene.

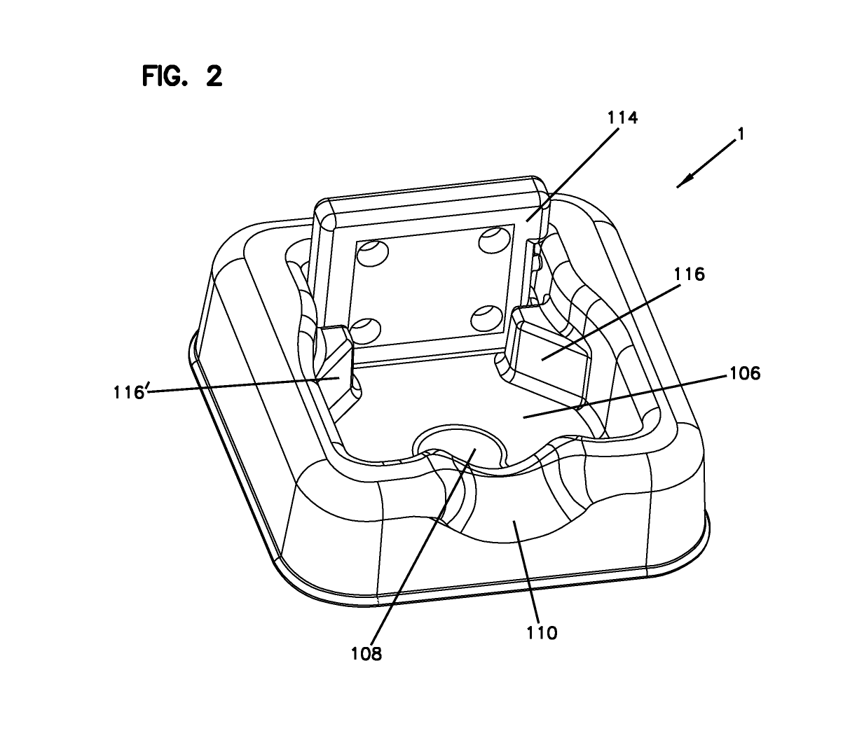

At least one receptacle 112 is formed within the container 102 and is separated from the cavity 106 by at least one divider 116. The receptacle 112 is configured to hold at least one cooling insert 114. The cooling insert 114 may serve as the cooling source to which heat is transferred from the head by indirect cooling through the liquid in the cavity 106. FIG. 2 shows one cooling insert 114 as it is placed in receptacle 112. In one embodiment, the receptacle 112 and dividers 116 and 116′ are located towards the back of the container 102 opposite the contour 110 and ramp 111 so that the cooling insert 114 is close to the top of the head when it is placed within the receptacle 112. Placing the receptacle 112 at the back of the container allows for efficient cooling to the top of the head. However, the receptacle(s) 112 can be located anywhere within the container so long as the head and/or neck are able to be cooled through the passive transfer of heat from the head to the cooling insert(s) 114 inside the receptacle(s) 112 through the liquid contained by the cavity 106. The cooling insert 114 may be made of plastic, rubber or other liquid resistant material. The cooling insert 114 may contain liquid for freezing or cooling, or other material capable of being frozen or cooled (e.g. cooling gel) including, for example, ice, gel or chemical inserts. The container 102 may have more than one receptacle 112 and cooling insert 114.

Generally, divider(s) 116 keep the cooling insert 114 away from direct contact with the head and at least partially partition receptacle 112 from the cavity 106. FIGS. 1-4 and 6-7 show two dividers 116 and 116′ placed at the back of the cavity. Dividers 116 and 116′ at least partially partition the receptacle 112 from the cavity 106. In one embodiment, the dividers 116 may form a bumper to keep the head from sliding into the receptacle 112. Dividers 116 can form a conduit for the transfer of heat by allowing direct contact of the liquid inside the cavity 106 with the cooling insert 114. FIGS. 1-4 and 6-7 show two dividers 116 and 116′ that are spaced apart from each other at the back of cavity 106. One divider 116′ is located on the left of the cavity and the other divider 116 is located on the right of the cavity. The space between dividers 116 and 116′ allows for the liquid inside the cavity to come into direct contact with the cooling insert 114 and serves as a conduit for the transfer of heat from the liquid inside the cavity 106 to the cooling insert 114. Any number of dividers 116 may be used as long as the divider(s) 116 allow the cooling insert(s) 114 to cool the liquid in the cavity 106 through the passive transfer of heat from the head through the liquid to the cooling insert 114. The divider(s) 116 may be injection molded and formed as a single structure with the container. In another embodiment the dividers may be formed separately from the container. In another embodiment, the dividers are removable. In another embodiment, the dividers may be padded so the head can rest against them. The divider(s) 116 may be made from any material as long as the material is capable of creating a space for the receptacle 112. In one embodiment, the dividers provide a conduit for the transfer heat from the liquid in the cavity 106 to the cooling insert 114. The divider may be made from mesh, plastic, resin or any other material capable of transferring heat. The direct contact between the intermediate medium and the cooling insert, allows for good heat transfer. The entire structure of the cooling apparatus can be integrally formed as a single unitary structure such as by a molding process.

In an alternative embodiment, the receptacle 112 may contain one or more stabilizing members 118 to hold the cooling insert 114 in place. FIGS. 1, 3-4 and 6 show two stabilizing members 118 and 118′ that are located at the back of the receptacle 112. Stabilizing member 118 may be disposed at the back of the receptacle 112 to allow the cooling insert 114 to be snap fitted or press fitted into place between the dividers and the stabilizing members. The stabilizing member 118 may be located anywhere inside the receptacle 112 as long as it is able to hold the cooling insert in place. The stabilizing member 118 may be a protrusion, snap or the like or any type of apparatus capable of stabilizing the detachable cooling insert 114 within the receptacle 112. However, it is appreciated that no stabilizing member is necessary.

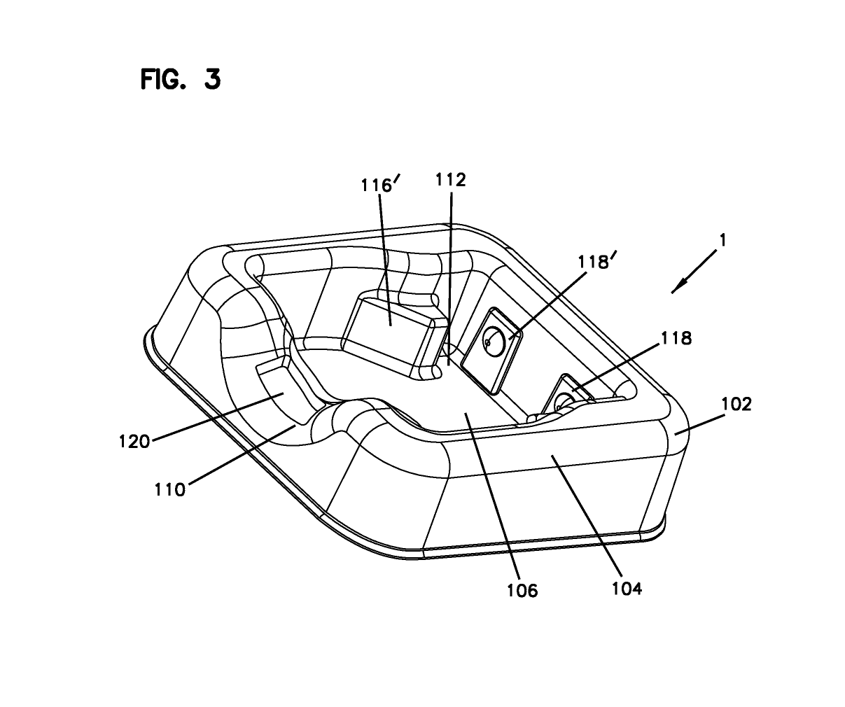

As described with reference to FIG. 3, the contour 110 of the cooling apparatus may contain a cut-away 120 into which a cooling apparatus (not shown) can be inserted to provide direct cool therapy to the neck. The cooling apparatus may be a gel, liquid or ice pack that is capable of following the contour of the neck and can be fitted into the cut away 120. In another embodiment, no cut away is formed in the contour and a cooling apparatus may be secured to the top of contour 102 by hook and loop attachments, straps, hooks, etc. The addition of a gel, liquid or ice pack could be used along with each of the embodiments discussed herein.

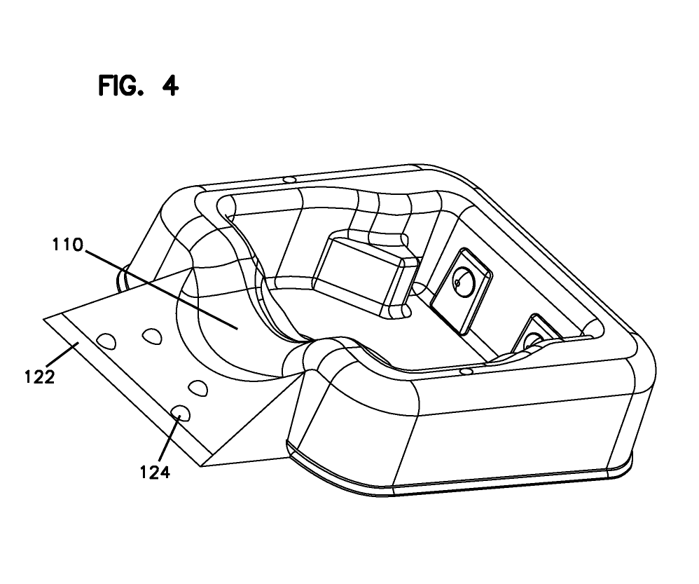

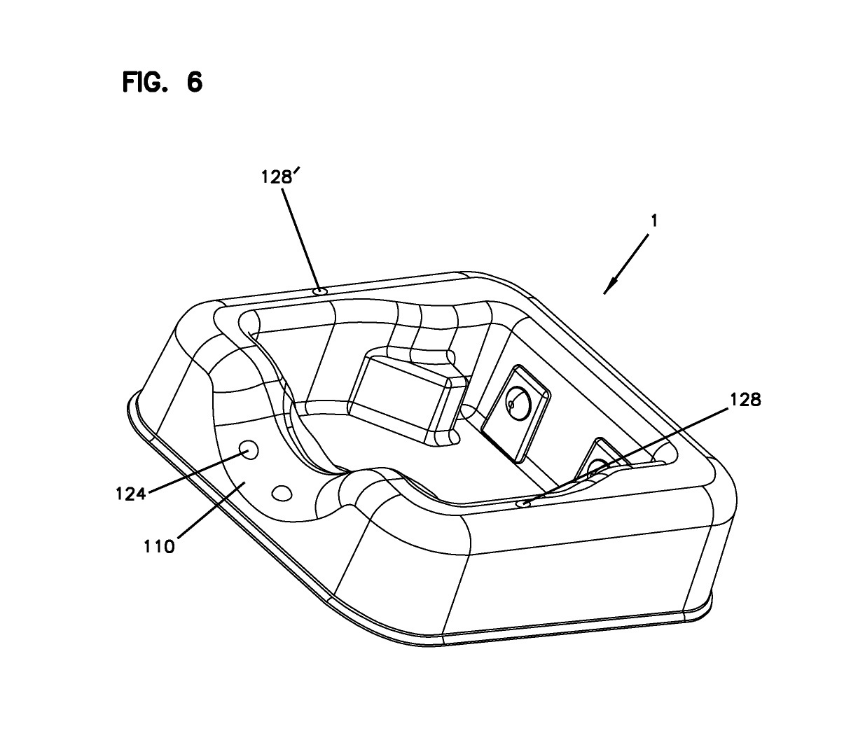

As described with reference to FIG. 4, a contour extension 122 may be added to contour 110 to extend outwards away from the container in a gentle slope to support the lower neck and upper shoulders when a person is lying on his back. The contour extension 122 may be padded with additional material and/or air to provide additional comfort for the back of the neck, back and shoulders. Protrusions 124 are shown on the contour extension 122 to provide additional pressure to trigger points along the neck and shoulders. Protrusions 124 may or may not be removable and are capable of being located anywhere on the cooling apparatus that may come into contact with the head, neck back and shoulders such as the contour 110, ramp 111 and indentation 108. The contour extension 122 may or may not be detachable from the neck contour 110. Protrusions 124 may exist in embodiments without the contour extension 122 such as shown in FIG. 6.

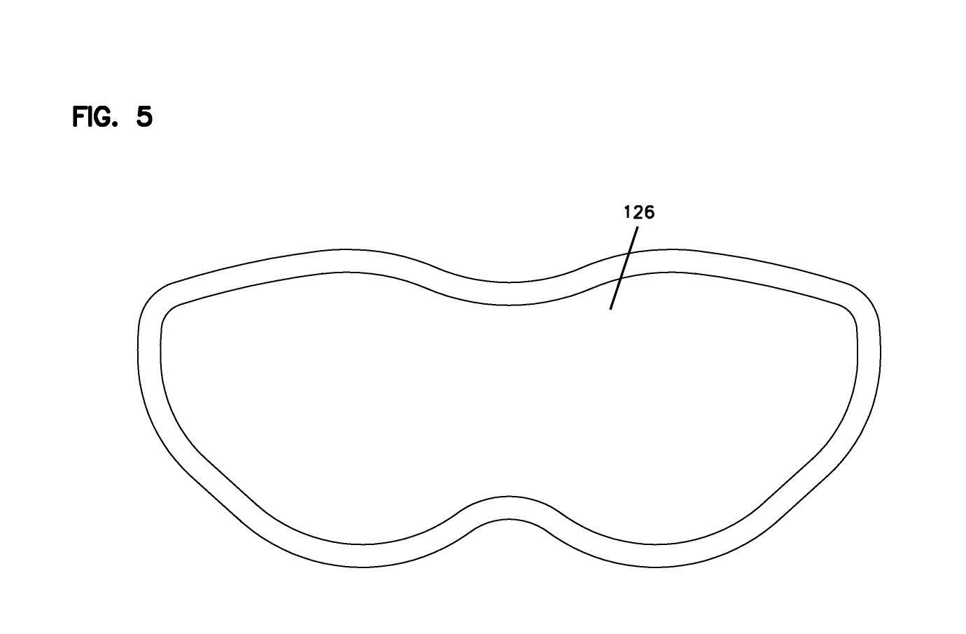

As described with reference to FIG. 5, an eye gel mask 126 is shown which can be used in tandem with the cooling apparatus as described with reference to FIGS. 1-4 and 6-7. The eye gel mask 126 may be filled with liquid or gel cooling material. The eye gel mask 126 may be cooled along with the cooling insert(s) 114 before using the cooling apparatus 1.

As described with reference to FIG. 6, the eye mask in FIG. 5 may be attached to eye gel mask attachment points 128 and 128′ so that the eye gel mask 126 is secured over a person's eyes when a person lies with their head in cavity 106. In another embodiment, the eye mask is not attached to the apparatus during use of the cooling device. In another embodiment, the eye gel mask is attached to attachment points 128 and 128′ when the cooling apparatus is stored between uses. They eye gel attachment points 128 and 128′ may comprise hook and loop material, snaps, protrusions, or any other attachment apparatus capable of securing the eye gel mask 126 to the cooling apparatus. The eye mask 126 attachment points may be adjustable according to the user of the eye gel mask.

As discussed above, protrusions 124 may be placed on contour 110, contour extension 122, ramp 111, and/or indentation 108 (or any other part of the apparatus that comes into contact with the head and/or neck) to apply pressure to trigger points on the neck, shoulders, head and/or back. Protrusions 124 may or may not be removable. Protrusions 124 are optional.

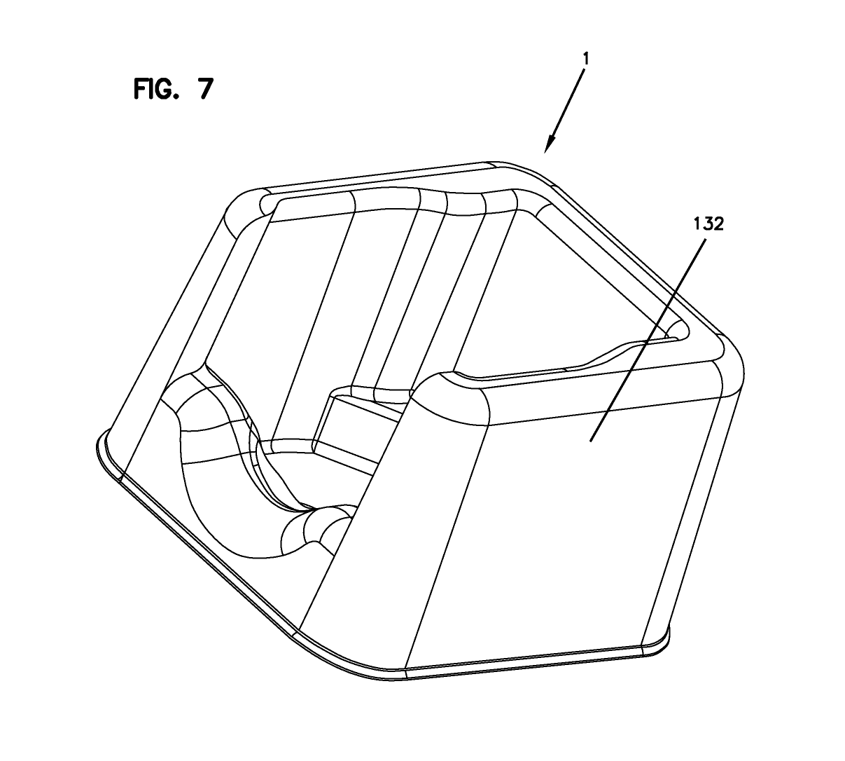

As described with reference to FIG. 7, a wall extension 132 is shown that extends above the cavity 106 to block additional light from the user of the apparatus 100. Additionally, a blanket or cover (not shown) may be placed on top of the apparatus to block all light from reaching the user of the apparatus. The wall extension 132 may be removable.

To use the apparatus, the cooling insert is cooled to a sufficient temperature. Non-limiting embodiments of cooling the cooling insert 114 include freezing and refrigerating. However, any cooling method will do. Once the insert 114 is sufficiently cooled, it is placed within the receptacle within the container 102. The cavity 106 may be filled with liquid before or after the cooling insert 114 is placed in the receptacle 112. Different amounts of liquid may be added to the cavity 106 as long as the liquid does not exceed the lowest point of the contour 110 (so that the liquid doesn't leak out). In one embodiment, the liquid level does not go above the ears. In another embodiment, approximately two quarts of liquid may be added to the cavity 106. A waterproof hairnet, e.g. a shower cap (not shown) may be used to prevent hair from getting wet. The liquid inside the cavity 106 may vary in initial temperature. For example, the initial liquid temperature may be lukewarm, room temperature or cool. After the cooling insert(s) 114 are added to the receptacle 112, the liquid inside the cavity 106 cools from its initial temperature over the next several minutes or longer. Heat is conducted from the head to the liquid or other intermediate medium, into the cooling insert 114. In one embodiment, the intermediate medium inside the cavity 106 is water such as water at room temperature, lukewarm water, or cold water, such as cold tap water.

A person positions his head in the cavity so that it is surrounded by liquid while the neck is supported by the contour 110 and ramp 111. The head and neck are gradually cooled by the passive transfer of heat from the head to the cooling insert 114 through the liquid. In addition, the person can place an eye gel mask 126 that has been cooled over the person's eyes while the head is cooled within the apparatus. In addition, the person may add a cooling apparatus to the contour 110 to apply direct cool therapy to the neck during treatment of the head with the cooling apparatus. Protrusions 124 on the ramp 111, indentation 108, contour 110 and/or contour extension 122 may provide additional stimulation to the head and neck.

With regard to the foregoing description, it is to be understood that changes may be made in detail, without departing from the scope of the present invention. It is intended that the specification and depicted embodiments are to be considered exemplary only, with a true scope and spirit of the invention being indicated by the broad meaning of the claims.

1. A therapeutic cooling apparatus comprising:
a container structure having at least one wall;
a cavity defined within the container structure;
a contour on the wall capable of supporting at least a portion of the head and neck;
an indentation on the bottom surface of the cavity;
a ramp that extends downwards from the contour to the indentation;
a receptacle configured to hold at least one cooling insert; and
at least one divider separating the receptacle from the cavity.

2. The apparatus of claim 1 in which the contour and indentation contain padding.

3. The apparatus of claim 1 in which the contour contains a cut-away into which a cooling gel can be inserted.

4. The apparatus of claim 1 further comprising protrusions to apply pressure to trigger points on the neck and head.

5. The apparatus of claim 1 in which the contour is extended to support the lower neck and upper shoulders.

6. The apparatus of claim 1 in which the cooling insert comprises an ice pack or gel pack.

7. The apparatus of claim 1 wherein at least one wall of the container extends upward from the cavity to block at least a portion of light from user's head when inside the cavity.

8. The apparatus of claim 1 further comprising an eye gel mask that is detachably connected to the housing.

9. The apparatus of claim 1 wherein the receptacle further comprises at least one stabilizing member.

10. A method for therapeutic cooling of a human head comprising:
cooling a cooling insert;
placing at least one cooling insert into a cooling apparatus having a receptacle, a cavity and a contour for supporting a neck;
filling the cavity with liquid;
positioning a head within the cavity so at least part of the head is in contact with the liquid while the neck is supported by the contour;
cooling the head by the passive transfer of heat from the head to the cooling insert through the liquid.

11. A method of making a therapeutic cooling apparatus comprising:
forming a container structure having at least one wall;
forming a cavity defined within the container structure;
forming a contour on the wall capable of supporting at least a portion of the head and neck;
forming an indentation on the bottom surface of the cavity;
forming a ramp that extends downwards from the contour to the indentation;
forming a receptacle configured to hold at least one cooling insert; and
forming at least one divider separating the receptacle from the cavity.
